(19) United States
(12) Patent Application Publication
(10) Pub. No.: US 2024/0021904 A1
(43) Pub. Date: Jan. 18, 2024

(54) RECYCLING METHOD AND USE OF LITHIUM IRON PHOSPHATE (LFP) WASTE

(71) Applicants: GUANGDONG BRUNP RECYCLING TECHNOLOGY CO., LTD., Foshan (CN); HUNAN BRUNP RECYCLING TECHNOLOGY CO., LTD., Changsha (CN); HUNAN BRUNP EV RECYCLING CO., LTD., Changsha (CN)

(72) Inventors: Jinliang Duan, Foshan (CN); Changdong Li, Foshan (CN); Yang Xia, Foshan (CN); Dingshan Ruan, Foshan (CN); Ruokui Chen, Foshan (CN); Yanchao Qiao, Foshan (CN)

(21) Appl. No.: 18/374,022

(22) Filed: Sep. 28, 2023

Related U.S. Application Data
(63) Continuation of application No. PCT/CN2021/142585, filed on Dec. 29, 2021.

(30) Foreign Application Priority Data
Apr. 29, 2021 (CN) 202110475141.9

(51) Int. Cl. H01M 10/54; C22B 1/00; C01D 15/00; C01G 49/16

(52) U.S. Cl. CPC H01M 10/54; C22B 1/005; C01D 15/00; C01G 49/16; C01P 2002/72; C01P 2004/03

(57) ABSTRACT

The present disclosure belongs to the technical field of battery recycling, and discloses a recycling method and use of lithium iron phosphate (LFP) waste. The method includes the following steps: mixing the LFP waste with water to prepare a slurry; adjusting a pH of the slurry to higher than 7.0 with an alkali, and heating to react; filtering a resulting mixture to obtain a filter residue; dissolving the filter residue in an acid, and filtering to obtain a filtrate; adding an oxalate-containing solution to react, and aging and filtering a resulting mixture to obtain a filter cake and a precipitation mother liquor; and subjecting the filter cake to slurrying, washing, and free water removal to obtain ferrous oxalate.

### CROSS-REFERENCE TO RELATED APPLICATIONS

The present application is a continuation application of PCT application No. PCT/CN2021/142585 filed on Dec. 29, 2021, which claims the benefit of Chinese Patent Application No. 202110475141.9 filed on Apr. 29, 2021. The contents of all of the aforementioned applications are incorporated by reference herein in their entirety.

### TECHNICAL FIELD

The present disclosure belongs to the technical field of battery recycling, and specifically relates to a recycling method and use of lithium iron phosphate (LFP) waste.

### BACKGROUND

LFP is considered to be the most promising new cathode material for lithium-ion batteries (LIB s) that is safe and environmentally-friendly, and LFP has high specific capacity, high stability, and prominent cycling performance and can be widely used in the fields of new energy vehicles, energy storage equipment, and etc.

At present, preparation methods of LFP mainly include high-temperature solid-phase reaction, microwave assisted synthesis, hydrothermal synthesis, sol-gel process, co-precipitation, etc. An iron source is a key raw material for preparing an LFP cathode material, and ferrous oxalate is one of the most common iron sources for the synthesis of LFP. The use of ferrous oxalate as an iron source has the following advantages: (1) an acid salt does not tend to introduce a miscellaneous phase during the synthesis of a cathode material; (2) an LFP cathode material synthesized from ferrous oxalate has high crystallinity and large bonding force, which helps to stabilize the framework structure of a sample; and (3) ferrous oxalate is decomposed to generate a gas during a reaction process, which can hinder the growth and agglomeration of crystal grains.

With the increasing use of batteries and the progressive realization of electric vehicle industrialization, the demand for ferrous oxalate will increase, and the quantity of scrapped LFP batteries will also increase. An existing LFP recycling method includes: dissolving an LFP battery cathode in an alkali, filtering a resulting mixture to obtain a filter residue, and dissolving the filter residue in a mixed acid liquor, such that iron exists in the form of an iron phosphate precipitate and is separated from impurities such as carbon black and a lithium-containing solution; and adding a 95° C. saturated sodium carbonate solution to the lithium-containing solution for precipitation to obtain lithium carbonate. The above-mentioned recycling method fails to achieve the efficient and high value-added recycling of LFP waste, and has the disadvantages of complicated and excessive process steps, large reagent consumption, and high cost.

Therefore, in order to solve the problems existing in waste battery treatment, there is an urgent need to develop a new battery waste treatment process.

### SUMMARY

The present disclosure is intended to solve at least one of the technical problems existing in the prior art. In view of this, the present disclosure provides a recycling method and use of LFP waste. The method can not only provide an iron source for the synthesis of LFP, but also relieve the pressure of waste battery treatment, which achieves the purpose of recycling and resource recovery and is of great practical significance for industrial production.

To achieve the above objective, the present disclosure adopts the following technical solutions:

The present disclosure provides a recycling method of lithium iron phosphate (LFP) waste, including the following steps:
(1) mixing the LFP waste with water to prepare a slurry; adjusting a pH of the slurry to higher than 7.0 with an alkali, and heating to react; and filtering a resulting mixture to obtain a filter residue;
(2) dissolving the filter residue in an acid, and filtering to obtain a filtrate; and adding an oxalate-containing solution to react, and aging and filtering a resulting mixture to obtain a filter cake and a precipitation mother liquor; and
(3) subjecting the filter cake to slurrying, washing, and free water removal to obtain ferrous oxalate.

Preferably, step (2) may further include adding a precipitating agent to the precipitation mother liquor for precipitation to obtain lithium dihydrogen phosphate; and the precipitating agent may be a lithium dihydrogen phosphate seed crystal.

After the seed crystal is added, evaporation can be conducted to increase the yield of lithium dihydrogen phosphate.

More preferably, before the precipitation mother liquor is subjected to precipitation, it may further include removing impurities from the precipitation mother liquor using an ion-exchange resin.

Preferably, in step (1), a solid-to-liquid ratio of the LFP waste to the water may be 1:(1-8) g/ml.

Preferably, in step (1), the alkali may be at least one from the group consisting of sodium hydroxide, ammonia water, and sodium carbonate; and the pH may be adjusted to 8.0 to 12.5.

Preferably, in step (1), the heating may be conducted at 25° C. to 80° C. for 30 min to 360 min.

Preferably, in step (2), the acid may be an inorganic acid; and the inorganic acid may be at least one from the group consisting of sulfuric acid, hydrochloric acid, nitric acid, and phosphoric acid. More preferably, the inorganic acid may be sulfuric acid or phosphoric acid.

In step (2), the acid may have an H⁺ concentration preferably of 0.5 mol/L to 18 mol/L and more preferably of 2 mol/L to 10 mol/L.

Preferably, in step (2), the oxalate-containing solution may be prepared as follows: dissolving an oxalate-containing substance in water, adding a surfactant, and stirring.

More preferably, the oxalate-containing substance may be at least one from the group consisting of oxalic acid, sodium oxalate, ammonium oxalate, and potassium oxalate.

In step (2), the oxalate-containing solution may have an oxalate concentration preferably of 5% to 50% and more preferably of 5% to 20%.

The surfactant may preferably be one or two from the group consisting of ethanol and 1-methyl-2-pyrrolidinone (NMP), and may more preferably be ethanol.

A mass ratio of the surfactant to the oxalate-containing substance may be preferably (0.05-1):1 and more preferably (0.1-0.8):1.

During a liquid phase reaction process, under special pH and solution composition conditions, a complicated oxalate complex will be formed. On the one hand, the addition of a surfactant can control the degree of hydrolysis (DH) of oxalic acid, thereby affecting an oxalate ion concentration in a solution. On the other hand, the addition of a surfactant can improve the surface energy of some crystal planes on the surface of a material, thereby regulating the purity, particle size, and morphology of the material. Therefore, the addition of a surfactant in an appropriate proportion can make synthesized ferrous oxalate have high crystallinity, high lattice stability, uniform particle dispersion, regular appearance, and no obvious impurities attached to the surface.

A molar ratio of Fe 2⁺ in the filtrate to C₂O₄²⁻ in the oxalate-containing solution may be preferably 1:(1-2.0) and more preferably 1:(1-1.3).

In step (2), the reaction may be conducted at a temperature preferably of 20° C. to 150° C. and more preferably of 25° C. and 80° C.; and the reaction may be conducted preferably for 10 min to 360 min and more preferably for 10 min to 120 min.

In step (2), the aging may be conducted preferably for 0.5 h to 24 h and more preferably for 1 h to 10 h. The oxalate-containing solution is continuously added to the iron solution; and after the oxalate-containing solution is completely added, stirring is stopped, and aging is conducted for a period of time. A reaction temperature and an aging time have a great impact on the quality of ferrous oxalate. The reaction temperature will affect the diffusion activation energy of an ionic reaction and thus affect the chemical reaction rate and the crystal nucleus growth rate, thereby regulating the morphology and purity of a material. In a preparation process of a material, the aging can promote the growth of crystal grains and the occurrence of secondary nucleation. An aging process is a process where the crystal shape becomes regular. A too-long aging time will cause the crystal grains to crack and destroy the crystal grain morphology. A too-short aging time will result in poor crystallinity of crystal grains and make it impossible to effectively control the particle morphology.

Preferably, in step (3), the free water removal may be conducted at 30° C. to 100° C.

Preferably, the filter cake may be washed to neutrality and then subjected to free water removal, and a drying temperature should not be too high, otherwise crystal water in the prepared ferrous oxalate will be removed.

The present disclosure also provides use of the recycling method described above in the preparation of an LFP cathode, a coating, or a ceramic.

Compared with the prior art, the present disclosure has the following beneficial effects.
1. In the present disclosure, an alkali is added to adjust a pH, and then a filter residue is dissolved in an acid; a resulting mixture is subjected to solid-liquid separation and a filter residue (graphite residue) is removed; and an oxalate-containing substance is added to a filtrate, and a resulting mixture is heated for precipitation to obtain a ferrous oxalate precipitate (recovered iron). Compared with the process of using LFP waste to synthesize iron phosphate, the process of using LFP waste to synthesize ferrous oxalate is easier to control and has a higher iron recovery rate (up to 99%). The ferrous oxalate prepared by the method of the present disclosure can be used as an iron source for the preparation of LFP cathode materials, and can also be used as a chemical raw material such as a colorant for coatings, ceramics, and the like.
2. In the method of the present disclosure, the precipitation mother liquor is subjected to impurity removal and precipitation to obtain lithium dihydrogen phosphate (recovery of lithium and phosphorus). The lithium dihydrogen phosphate prepared by the method is an important raw material for preparing a cathode material of an LFP power battery, and can also be used as a phosphorus source and a lithium source for preparing the LFP.
3. The recycling method of LFP waste provided by the present disclosure can not only provide an iron source for the synthesis of LFP, but also relieve the pressure of waste battery treatment. In the method, iron in the LFP waste is used to form ferrous oxalate, and the lithium and phosphorus are used to synthesize lithium dihydrogen phosphate, which realizes the efficient and high value-added recycling of LFP waste and is of great practical significance for industrial production.

### BRIEF DESCRIPTION OF DRAWINGS

FIG. 1 is a scanning electron microscopy (SEM) image of ferrous oxalate prepared in Example 1 of the present disclosure, with a magnification of 5,000;

FIG. 2 is an SEM image of ferrous oxalate prepared in Example 1 of the present disclosure, with a magnification of 50,000;

FIG. 3 is an SEM image of lithium dihydrogen phosphate prepared in Example 1 of the present disclosure, with a magnification of 1,000;

FIG. 4 is an SEM image of lithium dihydrogen phosphate prepared in Example 1 of the present disclosure, with a magnification of 5,000;

FIG. 5 is an X-ray diffractometry (XRD) pattern of ferrous oxalate prepared in Example 1 of the present disclosure; and

FIG. 6 is an XRD pattern of lithium dihydrogen phosphate prepared in Example 1 of the present disclosure.

### DETAILED DESCRIPTION

The concepts and technical effects of the present disclosure are clearly and completely described below in conjunction with examples, so as to allow the objectives, features and effects of the present disclosure to be fully understood. Apparently, the described examples are merely some rather than all of the examples of the present disclosure. All other examples obtained by those skilled in the art based on the examples of the present disclosure without creative efforts should fall within the protection scope of the present disclosure.

### Example 1

A recycling method of LFP waste was provided in this example, including the following steps:
(1) the LFP waste and water were mixed at a solid-to-liquid ratio of 5:1 and slurried, a sodium hydroxide liquid with a mass fraction of 30% was added to adjust a pH to 8.2, and a resulting slurry was heated at 60° C. for 120 min to react; and after the reaction was completed, a resulting mixture was filtered to obtain a filter residue;
(2) the filter residue was washed, dried, and then added to a 2 mol/L sulfuric acid solution; and a resulting mixture was stirred and reacted at 80° C. for 3 h and then subjected to liquid-solid separation, and a resulting filtrate (iron solution) was retained;
(3) oxalic acid was added to deionized water to prepare a 10% oxalic acid solution, and 10% of ethanol was added as a surfactant to dissolve oxalic acid;
(4) according to a Fe²⁺:C₂O₄²⁻ molar ratio of 1:1.3, the oxalic acid solution was continuously added to the iron solution for 2 h at 70° C. under stirring; after the oxalic acid solution was completely added, stirring was stopped and aging was conducted at 70° C. for 5 h; and a resulting mixture was subjected to liquid-solid separation to obtain a filter cake and a precipitation mother liquor;
(5) the obtained filter cake was washed with deionized water to neutrality, and then subjected to free water removal to obtain yellow ferrous oxalate; and
(6) a lithium dihydrogen phosphate seed crystal was added to the obtained precipitation mother liquor for precipitation to obtain lithium dihydrogen phosphate.

### Example 2

A recycling method of LFP waste was provided in this example, including the following steps:
(1) the LFP waste and water were mixed at a solid-to-liquid ratio of 3:1 and slurried, a sodium hydroxide liquid with a mass fraction of 30% was added to adjust a pH to 8.5, and a resulting slurry was heated at 55° C. for 150 min to react; and after the reaction was completed, a resulting mixture was filtered to obtain a filter residue;
(2) the filter residue was washed, dried, and then added to a 2 mol/L sulfuric acid solution; and a resulting mixture was stirred and reacted at 80° C. for 3 h and then subjected to liquid-solid separation, and a resulting filtrate (iron solution) was retained;
(3) oxalic acid was added to deionized water to prepare a 10% oxalic acid solution, and 10% of ethanol was added as a surfactant to dissolve oxalic acid;
(4) according to a Fe²⁺:C₂O₄²⁻ molar ratio of 1:1.3, the oxalic acid solution was continuously added to the iron solution for 2 h at 80° C. under stirring; after the oxalic acid solution was completely added, stirring was stopped and aging was conducted at 80° C. for 6 h; and a resulting mixture was subjected to liquid-solid separation to obtain a filter cake and a precipitation mother liquor;
(5) the obtained filter cake was washed with deionized water to neutrality, and then subjected to free water removal to obtain yellow ferrous oxalate; and
(6) a lithium dihydrogen phosphate seed crystal was added to the obtained precipitation mother liquor for precipitation to obtain lithium dihydrogen phosphate.

### Example 3

A recycling method of LFP waste was provided in this example, including the following steps:
(1) the LFP waste and water were mixed at a solid-to-liquid ratio of 5:1 and slurried, a sodium hydroxide liquid with a mass fraction of 20% was added to adjust a pH to 8.4, and a resulting slurry was heated at 65° C. for 150 min to react; and after the reaction was completed, a resulting mixture was filtered to obtain a filter residue;
(2) the filter residue was washed, dried, and then added to a 2 mol/L sulfuric acid solution; and a resulting mixture was stirred and reacted at 80° C. for 3 h and then subjected to liquid-solid separation, and a resulting filtrate (iron solution) was retained;
(3) oxalic acid was added to deionized water to prepare a 20% oxalic acid solution, and 10% of ethanol was added as a surfactant to dissolve oxalic acid;
(4) according to a Fe²⁺:C₂O₄²⁻ molar ratio of 1:1.2, the oxalic acid solution was continuously added to the iron solution for 2 h at 75° C. under stirring; after the oxalic acid solution was completely added, stirring was stopped and aging was conducted at 75° C. for 5 h; and a resulting mixture was subjected to liquid-solid separation to obtain a filter cake and a precipitation mother liquor;
(5) the obtained filter cake was washed with deionized water to neutrality, and then subjected to free water removal to obtain yellow ferrous oxalate; and
(6) a lithium dihydrogen phosphate seed crystal was added to the obtained precipitation mother liquor for precipitation to obtain lithium dihydrogen phosphate.

### Example 4

A recycling method of LFP waste was provided in this example, including the following steps:
(1) the LFP waste and water were mixed at a solid-to-liquid ratio of 5:1 and slurried, a sodium hydroxide liquid with a mass fraction of 10% was added to adjust a pH to 9.0, and a resulting slurry was heated at 70° C. for 180 min to react; and after the reaction was completed, a resulting mixture was filtered to obtain a filter residue;
(2) the filter residue was washed, dried, and then added to a 2 mol/L sulfuric acid solution; and a resulting mixture was stirred and reacted at 80° C. for 3 h and then subjected to liquid-solid separation, and a resulting filtrate (iron solution) was retained;
(3) oxalic acid was added to deionized water to prepare a 10% oxalic acid solution, and 10% of ethanol was added as a surfactant to dissolve oxalic acid;
(4) according to a Fe²⁺:C₂O₄²⁻ molar ratio of 1:1.3, the oxalic acid solution was continuously added to the iron solution for 1 h at 60° C. under stirring; after the oxalic acid solution was completely added, stirring was stopped and aging was conducted at 60° C. for 7 h; and a resulting mixture was subjected to liquid-solid separation to obtain a filter cake and a precipitation mother liquor;
(5) the obtained filter cake was washed with deionized water to neutrality, and then subjected to free water removal to obtain yellow ferrous oxalate; and
(6) a lithium dihydrogen phosphate seed crystal was added to the obtained precipitation mother liquor for precipitation to obtain lithium dihydrogen phosphate.

### Comparative Example 1

A recycling method of LFP waste was provided in this comparative example, including the following steps:
(1) the LFP waste and water were mixed at a solid-to-liquid ratio of 5:1 and slurried, a sodium hydroxide liquid with a mass fraction of 30% was added to adjust a pH, and a resulting slurry was heated and reacted for a specified time; and after the reaction was completed, a resulting mixture was filtered to obtain a filter residue;
(2) the filter residue was washed, dried, and then added to a 2 mol/L mixed acid solution of sulfuric acid and hydrochloric acid; and a resulting mixture was stirred and reacted at 80° C. for 3 h and then subjected to liquid-solid separation to obtain iron phosphate and a filtrate; and
(3) a 95° C. saturated sodium carbonate was added to the obtained filtrate, such that lithium was precipitated in the form of a lithium carbonate solid; and the filter residue was added to hydrochloric acid (iron was dissolved into the solution in the form of ions, thereby achieving the separation of iron from solid impurities), a resulting mixture was stirred at 50° C. for 6 h and then filtered to obtain a filtrate, and a pH of the filtrate was adjusted with NaOH+ammonia water to obtain iron hydroxide.

Result Comparison:
(1) The recovery rates of iron from LFP waste in Examples 1 to 2 were compared with that in Comparative Example 1, separately.

TABLE 1
Iron recovery rate

| | Example 1 | Example 2 | Comparative Example 1 |
|---|---|---|---|
| Iron recovery rate (%) | 99.02 | 99.10 | 98.30 |

It can be seen from Table 1 that, compared with the process of using LFP waste to synthesize iron phosphate, the process of using LFP waste to synthesize ferrous oxalate is easier to control and has a higher iron recovery rate.

TABLE 2
Lithium dihydrogen phosphate

| | Example 1 | Example 2 | Example 3 |
|---|---|---|---|
| Phosphorus recovery rate (%) | 96.73 | 97.56 | 96.98 |
| Lithium recovery rate (%) | 96.92 | 97.79 | 97.12 |

It can be seen from the data in Table 2 that, during the process of adding a precipitating agent to the precipitation mother liquor obtained after iron is precipitated for precipitation to obtain lithium dihydrogen phosphate in the present disclosure, the recovery rates of phosphorus and lithium are both greater than 95%, indicating prominent recovery effect.

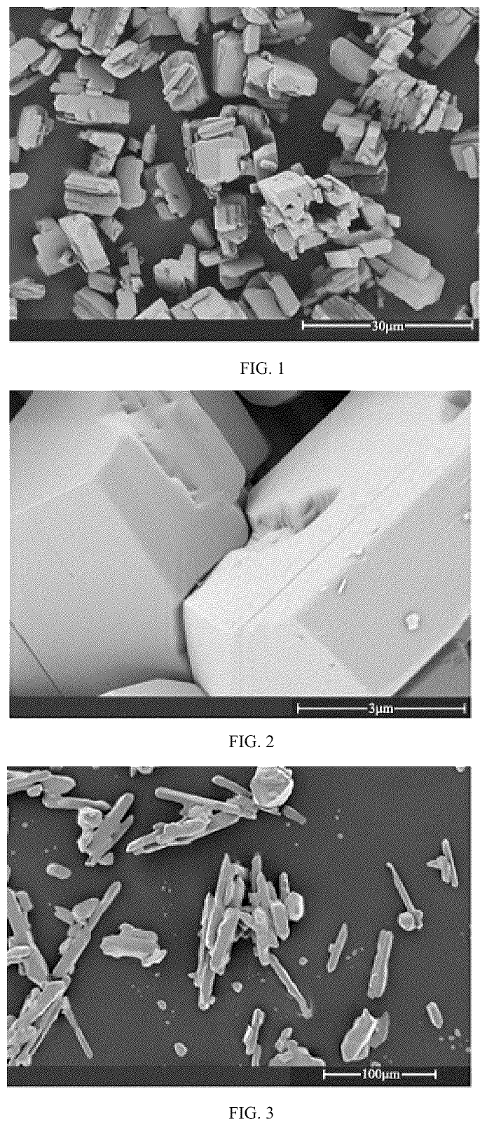

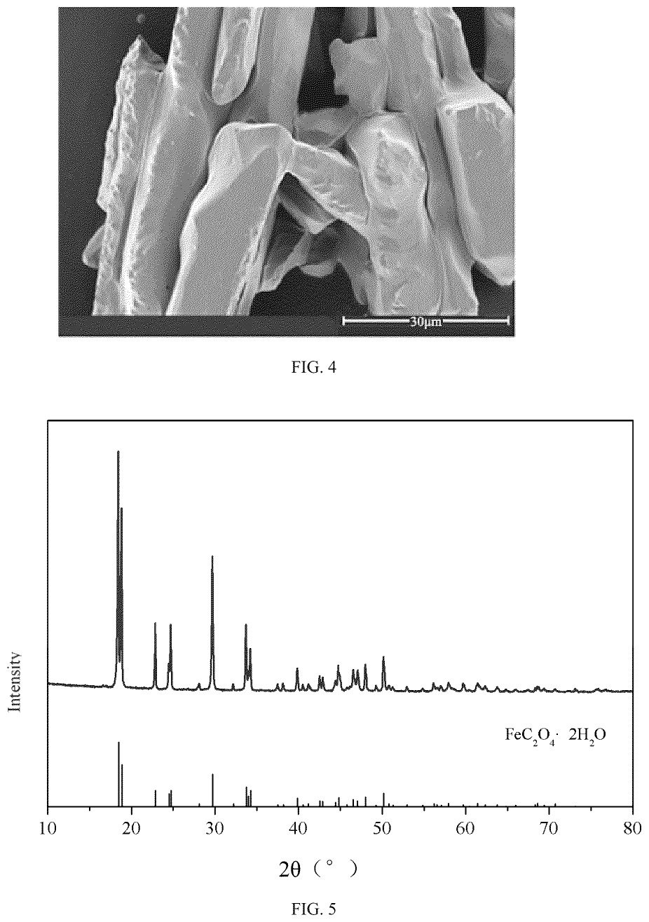

FIG. 1 and FIG. 2 show SEM images of ferrous oxalate prepared in Example 1 of the present disclosure. It can be seen from FIG. 1 and FIG. 2 that the synthesized ferrous oxalate has a block structure with a smooth surface, a uniform particle distribution, and a particle size of 8 μm to 10 μm. FIG. 3 and FIG. 4 show SEM images of lithium dihydrogen phosphate prepared from the precipitation mother liquor in Example 1 of the present disclosure. It can be seen from FIG. 3 that the lithium dihydrogen phosphate has needle-like and rod-like structures, and it can be seen from FIG. 4 that the needle-like and rod-like structures in the lithium dihydrogen phosphate are interleaved with each other and have a smooth surface.

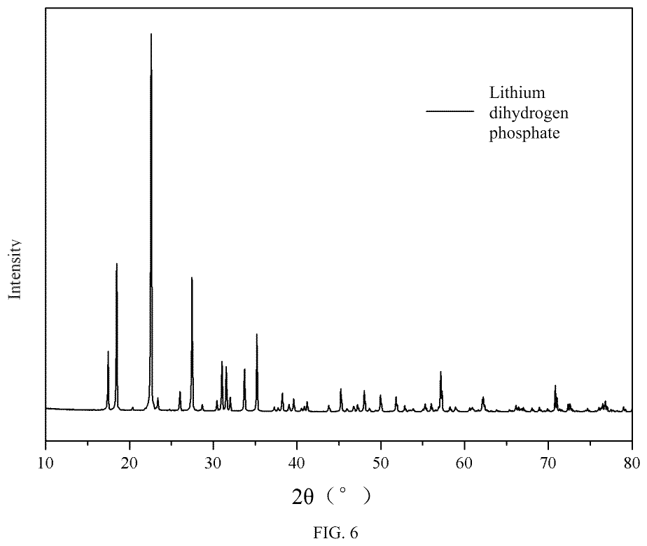

FIG. 5 shows an XRD pattern of ferrous oxalate prepared in Example 1 of the present disclosure. It can be seen from FIG. 5 that the characteristic peaks in the XRD pattern of the prepared ferrous oxalate are corresponding to that in the spectrum of standard card (23-0293), respectively; and the diffraction peaks are sharp, characteristic peaks are obvious, and there are no impurity peaks, indicating that ferrous oxalate with high crystallinity is obtained. FIG. 6 shows an XRD pattern of lithium dihydrogen phosphate prepared in Example 1 of the present disclosure. Through comparative analysis and literature review, it can be known that the crystal phase of the material is formed, the characteristic peaks are obvious, and the space group is Pna21, indicating that the prepared lithium dihydrogen phosphate has prominent crystallinity and high purity.

The examples of present disclosure are described in detail with reference to the accompanying drawings, but the present disclosure is not limited to the above examples. Within the scope of knowledge possessed by those of ordinary skill in the technical field, various changes can also be made without departing from the purpose of the present disclosure. In addition, the examples in the present disclosure and features in the examples may be combined with each other in a non-conflicting situation.

1. A recycling method of lithium iron phosphate (LFP) waste, comprising the following steps:
(1) mixing the LFP waste with water to prepare a slurry; adjusting a pH of the slurry to higher than 7.0 with an alkali, and heating to react; and filtering a resulting mixture to obtain a filter residue;
(2) dissolving the filter residue in an acid, and filtering to obtain a filtrate; and adding an oxalate-containing solution to allow a reaction to take place, and aging and filtering a resulting mixture to obtain a filter cake and a precipitation mother liquor; and
(3) subjecting the filter cake to slurrying, washing, and free water removal to obtain ferrous oxalate;
wherein step (2) further comprises adding a precipitating agent to the precipitation mother liquor for precipitation to obtain lithium dihydrogen phosphate; and the precipitating agent is a lithium dihydrogen phosphate seed crystal;
in step (2), the acid is an inorganic acid; and the inorganic acid is at least one from the group consisting of sulfuric acid, hydrochloric acid, nitric acid, and phosphoric acid;
in step (2), the oxalate-containing solution is prepared as follows: dissolving an oxalate-containing substance in water, adding a surfactant, and stirring, the oxalate-containing substance is oxalic acid; the surfactant is one or two from the group consisting of ethanol and 1-methyl-2-pyrrolidinone (NMP).

2. The recycling method according to claim 1, wherein in step (1), main components of the LFP waste are LiFePO₄ and C.

3. The recycling method according to claim 1, wherein in step (1), the alkali is one or two from the group consisting of sodium hydroxide, ammonia water; and the pH is adjusted to 8.0 to 12.5.

4. The recycling method according to claim 1, wherein in step (2), the reaction is conducted at 20° C. to 150° C. for 10 min to 360 min; and the aging is conducted for 0.5 h to 24 h.
